(19) United States
(12) Patent Application Publication
(10) Pub. No.: US 2008/0108890 A1
(43) Pub. Date: May 8, 2008

(54) PHYSIOLOGICAL DATA MEASURING APPARATUS AND MEASURING STRIP

(75) Inventors: CHYN NAN TENG, KAOHSIUNG CITY (TW); YANG-WEI WU, TAIPEI COUNTY (TW)

Correspondence Address: ROSENBERG, KLEIN & LEE, 3458 ELLICOTT CENTER DRIVE-SUITE 101, ELLICOTT CITY, MD, 21043, US

(73) Assignee: LEADTEK RESEARCH INC., TAIPEI HSIEN (TW)

(21) Appl. No.: 11/751,266

(22) Filed: May 21, 2007

(30) Foreign Application Priority Data
Nov. 7, 2006 (TW) 095141220

(51) Int. Cl. A61B 5/04

(52) U.S. Cl. 600/372

(57) ABSTRACT

A physiological data measuring apparatus and a measuring strip are disclosed. The apparatus includes a measuring body and a measuring strip. The measuring body reads, displays and records a physiological signal. The measuring strip has a containing space for accommodating the measuring body and includes conducting metals for measuring and transmitting the physiological signal to the measuring body, wherein the conducting metals constitute a set of paired conducting metals that are wrapped by a soft material of the measuring strip. The soft material forms the containing space and two wings, and a pair of conducting metals are disposed at the bottom of the containing space and penetrates through the soft material to form two contact points, and another pair of conducting metals is disposed at the two wings outside the containing space to form a third contact point. The strip includes at least one adhesive for attaching a live organism.

### BACKGROUND OF THE INVENTION

1. Field of the Invention

The present invention relates to a physiological data measuring apparatus and a measuring strip, and more particularly to a measuring strip made of a soft material and installed to a measuring body to constitute a physiological data measuring apparatus.

2. Description of the Related Art

Electrocardiography is a necessary tool for providing reference data for a medical treatment of cardiovascular diseases, and most of the present electrocardiograms are that a large measuring instrument is utilized to connect a plurality of external electrodes attached to the positions such as an examinee's chest, heart and different positions of the body to read an electrocardiograph. However, these electrocardiograms are huge and inconvenient-to-carry. A general user who has no professional training may find these instruments difficult to operate. Furthermore, there are many external electrodes to be attached onto an examinee's body, and thus improvements are developed. Referring to FIG. 1 for a conventional electrocardiograms, a liquid crystal display 11 is installed on the surface of a body 1 for displaying information, and three circular electrode touch keys 12 are disposed under both left and right sides and the bottom of the display device 11, and a plurality of function keys 13˜17 are disposed sequentially at the middle of the bottom of the display device 11. The electrocardiograms comes with simple electrodes, and simply requires users to touch the three electrode touch keys by the thumb and index fingers of both hands or connect external electrode connection plugs for measuring a static or dynamic electrocardiograph, and thus a non-professional user can operate the device easily.

However, the foregoing devices still require external electrodes, and may not be able to make the measurement successfully if the cuticle of an examinee's finger is thick. Furthermore, the volume of the device is still not portable or convenient to carry, and thus these conventional devices still require further improvements.

In view of shortcomings of the prior art, the inventor of the present invention based on years of experience in the medical measuring apparatus related industry to conduct extensive researches and experiments, and finally developed a physiological data measuring apparatus and a measuring strip in accordance with the present invention to overcome the aforementioned shortcomings.

### SUMMARY OF THE INVENTION

Therefore, it is a primary objection of the present invention to provide a physiological data measuring apparatus and a measuring strip and more particularly to a measuring strip made of a soft material and installed to a measuring body to constitute a physiological data measuring apparatus.

To achieve the foregoing objective, the present invention provides a physiological data measuring apparatus comprising a measuring body and a measuring strip. The measuring body is provided for reading, displaying and recording a physiological signal. The measuring strip has a containing space for accommodating the measuring body and includes a plurality of conducting metals for measuring a physiological signal and transmitting the physiological signal to the measuring body.

Further, the measuring strip of the invention is applied to a physiological data measuring apparatus, and the measuring strip comprises a set of paired conducting metals and a soft material. The soft material is provided for wrapping the set of paired conducting metals, and forming a containing space and at least two wings, wherein a pair of conducting metals in the set of conducting metals are disposed at the bottom of the containing space and penetrate through the soft material, and another pair of conducting metals are disposed at two wings outside the containing space.

Since the physiological data measuring apparatus and the measuring strip of the invention can be miniaturized to achieve an easy-to-carry effect or attached directly onto the surface of human skin such as a position around an examinee's chest, heart or forehead without requiring additional external electrodes so as to measure, display or store related physiological signals.

To make it easier for our examiner to understand the technical characteristics and effects of the present invention, we use preferred embodiments together with the attached drawings for the detailed description of the present invention.

### BRIEF DESCRIPTION OF THE DRAWINGS

FIG. 1 shows a conventional electrocardiograms;

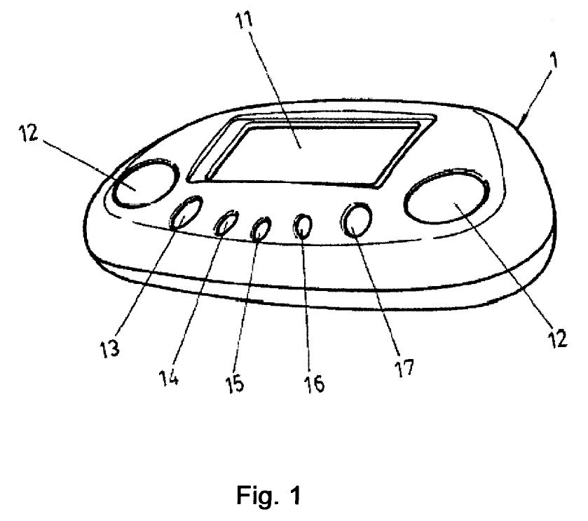

FIG. 2 is a top view of a measuring strip of the present invention;

FIG. 3 is a cross-sectional view of a measuring strip of the present invention;

FIG. 4 is a schematic view of a physiological data measuring apparatus of the present invention;

FIG. 5 is a perspective view of an inverted physiological data measuring apparatus of the present invention; and

FIG. 6 is a cross-sectional view of a physiological data measuring apparatus of the present invention.

### DETAILED DESCRIPTION OF THE PREFERRED EMBODIMENTS

In describing a preferred embodiment of a physiological data measuring apparatus and a measuring strip in accordance with the invention illustrated in the drawings, specific terminologies and numerals will be resorted for the sake of clarity. However, the invention is not intended to be limited to the specific terms and numerals so selected, and it is to be understood that each specific term includes all technical equivalents which operate in a similar manner to accomplish a similar purpose.

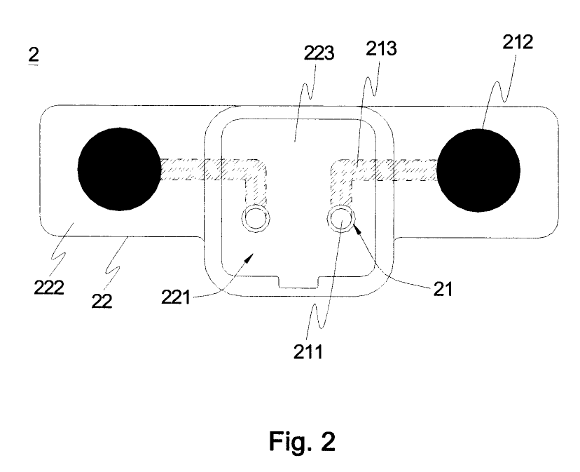

Referring to FIG. 2 for a top view of a measuring strip of the present invention, the measuring strip 2 is applied to a physiological data measuring apparatus, and the measuring strip 2 comprises symmetric paired conducting metals 21 and a soft material 22, and the soft material 22 is used for wrapping the paired conducting metals 21 to define a containing space 221 and at least two wings 222, wherein each of the paired conducting metals 21 includes three parts of conducting metals 211, 212 and a thin conducting plate 213. The conducting metal 211 is disposed at the bottom 223 of the containing space 221 and penetrates through the soft material 22, and another pair of conducting metals 212 are disposed on two wings 222 outside the containing space 221 and embedded into the soft material 22, and both conducting metals 211, 212 are connected through the thin conducting plate 213.

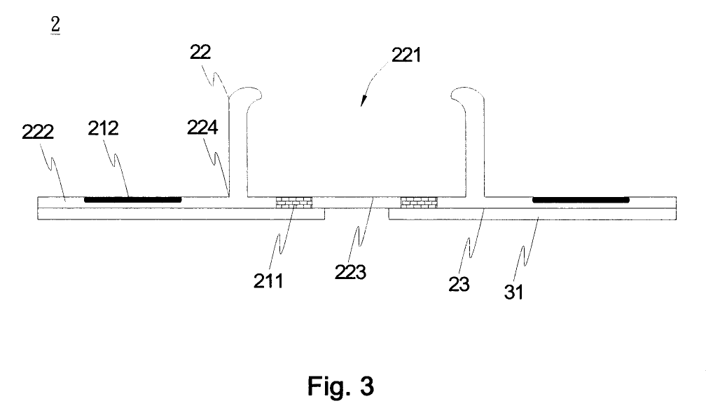

Referring to FIG. 3 for a cross-sectional view of a measuring strip of the present invention, the measuring strip 2 comprises a set of paired conducting metals (not shown in the figure) and a soft material 22, and the soft material 22 is provided for wrapping the set of paired conducting metals (not shown in the figure) and forming a containing space 221 and at least two wings 222, and the containing space 221 is substantially in a square shape, and two wings 222 formed on two corresponding sides 224 and the bottom 223 are on the same plane, wherein a pair of conducting metals 211 in the set of paired conducting metals (not shown in the figure) are disposed at a bottom 223 of the containing space 221 and penetrates through a soft material 22, and another pair of conducting metals 212 are disposed on two wings 222 outside the containing space 221 and embedded into the two wings 222 of the soft material 22, and the bottom surface 23 of the measuring strip 2 further includes at least one conducting adhesive 31 attached completely to the bottom surface 23 and contacted with the conducting metal 211 for attaching a live organism.

The set of paired conducting metals 21 of the measuring strip 2 as shown in FIGS. 2 and 3 form at least three contact points on the soft material 22, wherein a contact point is situated at the bottom of the containing space 221 and penetrates through the soft material 22 to the conducting metal 211 at the bottom surface 23 of the measuring strip 2, and a contact point is formed at the bottom of the containing space 221 and the bottom surface of the measuring strip 2, and a contact point is formed at two wings 222 of the conducting metal 212 extended by the thin conducting plate 213, and the soft material is preferably silicon, and the conducting metal is preferably chrome or stainless steel, and the conducting adhesive is preferably a replaceable conducting adhesive, and the physiological data measuring apparatus preferably measures a physiological signal such as an electrocardiographic signal and/or a temperature signal.

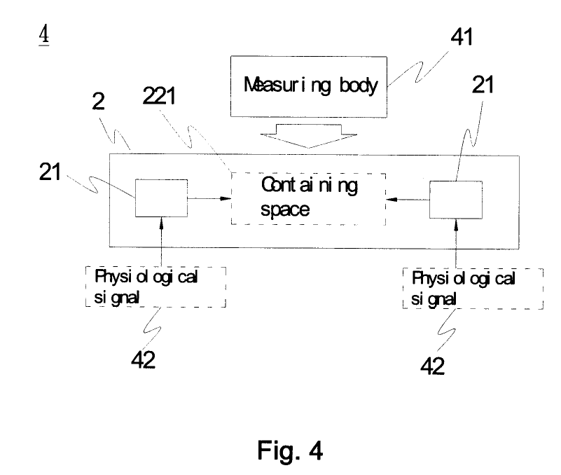

Referring to FIG. 4 for a schematic view of a physiological data measuring apparatus of the present invention, the physiological data measuring apparatus 4 comprises a measuring body 41 and a measuring strip 2, and the measuring body 41 is provided for reading, displaying or recording a physiological signal 42, and the measuring strip 2 has a containing space 221 for accommodating the measuring body 41 and includes a plurality of paired conducting metals 21 for measuring the physiological signal 42 and transmitting the physiological signal 42 to the measuring body 41.

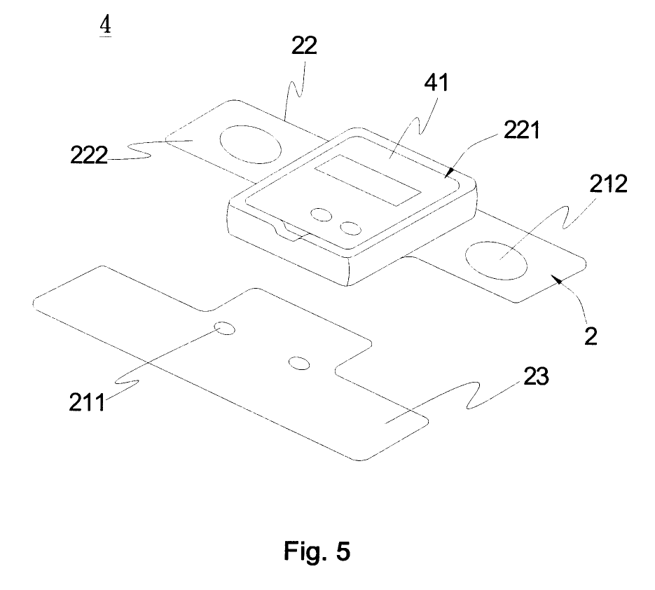

Referring to FIG. 5 for a perspective view of an inverted physiological data measuring apparatus, the physiological data measuring apparatus 4 comprises a measuring body 41 and a measuring strip 2, wherein the measuring body 41 is accommodated in the containing space 221 of the measuring strip 2, and a pair of conducting metals 212 are embedded into the two wings 222 to provide a user to measure a physiological signal 42 by a touch of finger, and a conducting metal 211 is disposed at the bottom surface 23 of the measuring strip 2 and penetrated from the bottom 223 of the containing space 221 and provided for a user to attach the measuring strip 2 to a user's desired measuring position of the skin in order to measure a physiological signal 42 by the physiological data measuring apparatus 4. These measuring positions include the user's chest and heart for measuring an electrocardiographic signal and/or a body temperature, or the user's forehead for measuring a body temperature.

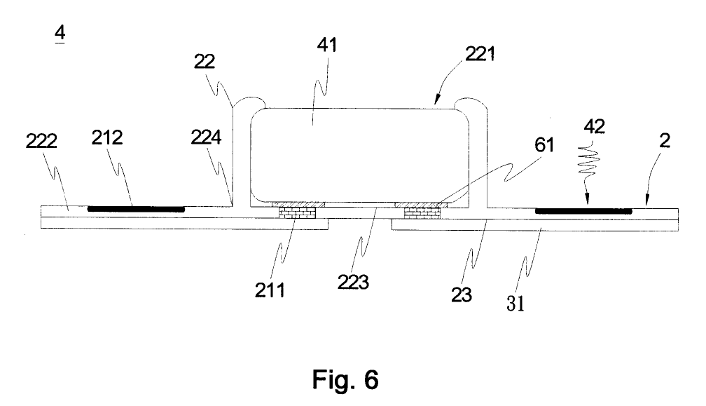

Referring to FIG. 6 for a cross-sectional view of a physiological data measuring apparatus of the present invention, the physiological data measuring apparatus 4 comprises a measuring body 41 and a measuring strip 2, wherein the measuring body 41 is provided for reading, displaying or recording a physiological signal 42, and the measuring strip 2 has a containing space 221 and two wings 222, and the containing space 221 is provided for accommodating the measuring body 41, and the two wings 222 are formed on both corresponding sides 224 of the containing space 221, and on the same plane together with the bottom 223, wherein a pair of conducting metals 211 in the set of conducting metals 21 are disposed at the bottom 223 of the containing space 221 and penetrates through the soft material 22 of the measuring strip 2 and contacted with the conducting metal 61 of the measuring body 41 for receiving a physiological signal 42, and the conducting metal 61 is protruded slightly from the surface of a measuring body 41, or embedded into the measuring body 41, and the exposed portion and the measuring body 41 are on the same plane. FIG. 6 is provided for illustration only. Another pair of conducting metals 212 are situated at and embedded into two wings 222 outside the containing space 221. The bottom surface 23 of the measuring strip 2 further includes at least one conducting adhesive 31 for attaching a live organism.

In FIGS. 4 to 6, the soft material is preferably silicon, and the conducting metal is preferably chrome or stainless steel, and the conducting adhesive is preferably a replaceable conducting adhesive, and the physiological signal is preferably an electrocardiographic signal and/or a temperature signal.

While the invention has been described by means of specific embodiments, numerous modifications and variations could be made thereto by those skilled in the art without departing from the scope and spirit of the invention set forth in the claims.

What is claimed is:

1. A measuring strip, applied in a physiological data measuring apparatus, and said measuring strip comprising:
at least one paired conducting metals; and
a soft material for wrapping said paired conducting metals, and forming a containing space and at least two wings;
wherein a pair of conducting metals of said paired conducting metals are disposed at the bottom of said containing space and penetrates through said soft material, and another pair of conducting metals are disposed at said wings outside said containing space.

2. The measuring strip of claim 1, wherein said containing space defined by said soft material is substantially in a square shape, and said wings formed at both corresponding sides and said bottom are on the same plane.

3. The measuring strip of claim 1, wherein said paired conducting metals form at least three contact points on said soft material.

4. The measuring strip of claim 1, wherein the measuring strip further comprising at least one conducting adhesive for adhering a live organism.

5. The measuring strip of claim 4, wherein said conducting adhesive is a replaceable conducting adhesive.

6. The measuring strip of claim 1, wherein said soft material is silicon.

7. The measuring strip of claim 1, wherein said conducting metal is chrome or stainless steel.

8. The measuring strip of claim 1, wherein said physiological data measuring apparatus measures an electrocardiographic signal, a temperature signal or both.

9. A physiological data measuring apparatus, comprising:
a measuring body for reading, displaying and recording at least one physiological signal; and
a measuring strip having a containing space for accommodating said measuring body, and including a plurality of conducting metals for measuring said physiological signal to be transmitted to said measuring body.

10. The physiological data measuring apparatus of claim 9, wherein said measuring body comprises two conducting metals contacted with said measuring strip for receiving said physiological signals.

11. The physiological data measuring apparatus of claim 9, wherein said measuring strip is made of a soft material for wrapping said conducting metals.

12. The physiological data measuring apparatus of claim 9, wherein said measuring strip is coated with at least one conducting adhesive for attaching a live organism.

13. The physiological data measuring apparatus of claim 12, wherein said conducting adhesive is a replaceable conducting adhesive.

14. The physiological data measuring apparatus of claim 11, wherein said soft material is silicon.

15. The physiological data measuring apparatus of claim 9, wherein said conducting metal is chrome or stainless steel.

16. The physiological data measuring apparatus of claim 9, wherein said physiological signal is an electrocardiographic signal, a temperature signal or both.
